(19) United States
(12) Patent Application Publication
(10) Pub. No.: US 2012/0316638 A1
(43) Pub. Date: Dec. 13, 2012

(54) METHOD AND DEVICE FOR TREATING CEREBROVASCULAR PATHOLOGIES AND DELIVERY SYSTEM THEREFOR

(75) Inventors: Ygael Grad, Tel-Aviv (IL); Marc Litzenberg, Miami Beach, FL (US)

(73) Assignee: Surpass Medical Ltd., Tel-Aviv (IL)

(21) Appl. No.: 13/576,663

(22) PCT Filed: Jan. 26, 2011
(86) PCT No.: PCT/IL11/00091

§ 371 Date: Aug. 2, 2012

Related U.S. Application Data
(60) Provisional application No. 61/302,203, filed on Feb. 8, 2010.

(51) Int. Cl. A61F 2/84

(52) U.S. Cl. 623/1.12; 623/1.11

(57) ABSTRACT

In one embodiment, a delivery system for a medical device includes a flexible sheath defining a lumen having a distal end and a flexible shaft having a proximal portion and a distal portion. A retention element and a pushing element, spaced proximally from the retention element, are disposed on the distal portion. The flexible shaft, retention element, and pushing element are disposable within the lumen. The flexible shaft is movable distally through the lumen between a stowed position and a deployed position and is movable proximally through the lumen between the deployed position and a partially deployed position with the retention element proximate to the distal end of the lumen. The retention element is configured to form with the medical device an interference fit with the flexible sheath when the flexible shaft is urged proximally from the retention position to engage the medical device with the flexible sheath.

### BACKGROUND

The invention relates generally to medical devices and procedures, including, for example, medical devices and methods for delivering a medical implant to a treatment site.

A cerebral aneurysm is a weak region in the wall of an artery in the brain where dilation or ballooning of the artery wall may occur. Cerebral aneurysms are common lesions and a rupture can lead to fatal hemorrhages in the brain. A known endovascular treatment of cerebral aneurysms includes the use of flow diverting devices that can provide the main engine for arterial remodeling and vascular tissue growth.

Some known methods for treating intracranial aneurysms include surgical clipping and endovascular coiling. In the surgical clipping method, the skull of the patient is opened, and a surgical clip is placed across the neck of the aneurysm to stop blood from flowing into the aneurysm sac. An endovascular coiling procedure is a less invasive method involving placement of one or more coils, delivered through a catheter, into the aneurysm until the sac of the aneurysm is completely packed with coils. It also helps to trigger a thrombus inside the aneurysm. In a stent-assisted coiling procedure, a stent is first placed across the aneurysm neck, serving as a scaffold inside the lumen and then the coils are delivered into the sac of the aneurysm through the interstices of the stent.

In another known method of treatment, a tubular porous device is placed in the artery harboring the aneurysm to treat the aneurysm. In this method, a tubular porous device with a sufficient material coverage is placed across the aneurysm neck, blocking it sufficiently to restrain blood from flowing into the sac and to trigger a thrombus within the aneurysm. Because the aneurysm thrombus in the aneurysm solidifies naturally on itself and is subsequently converted to scar tissue, ruptures may be reduced or eliminated. Furthermore, because no coil is involved in this method, the aneurysm will gradually shrink as the thrombus is absorbed. Consequently, the pressure applied on the aneurysm sac can be reduced.

With many of the known delivery devices for such intraluminal devices, such as flow diverters and stents, mechanical fasteners are used to secure the intraluminal device to the delivery device, which can lead to possible complications in the delivery process. In addition, with existing systems, once the deployment of the intraluminal device begins at a particular location within an intracranial blood vessel, the intraluminal device cannot be retracted or moved to a different location.

Thus, a need exist for a delivery system that can deliver such intraluminal medical devices without the need for mechanically fastening the intraluminal device to the delivery device. A need also exist for a delivery system that allows for the recapture and repositioning of an intraluminal device within a cerebral blood vessel, which are typically very tortuous.

### SUMMARY OF THE INVENTION

Devices and methods for treating and/or diagnosing blood vessels, such as cerebral blood vessels are disclosed herein. In one embodiment, a delivery system for a medical device includes a flexible sheath having an interior wall bounding a lumen having a distal end and a flexible shaft having a proximal portion and a distal portion. A retention element and a pushing element are each disposed on the distal portion. The pushing element is spaced proximally from the retention element. The flexible shaft, retention element, and pushing element are disposable within the lumen to collectively define with the interior wall a region sized to contain the medical device. The flexible shaft is movable distally through the lumen between a stowed position in which the retention element is proximal to the distal end of the lumen and a deployed position in which the pushing element at least partially extends distally from the distal end of the lumen. The flexible shaft is movable proximally through the lumen between the deployed position and a partially deployed position in which the retention element is proximate to the distal end of the lumen. The retention element is configured to form with the medical device an interference fit with the flexible sheath when the flexible shaft is urged proximally from the partially deployed position to a retention position to engage the medical device with the flexible sheath.

### BRIEF DESCRIPTION OF THE DRAWINGS

FIG. 1 is a schematic illustration of a delivery system according to an embodiment.

FIG. 2 is a side view shown partially in cross-section of a portion of a delivery system according to an embodiment.

FIG. 3 is a side view shown partially in cross-section of a portion of the delivery system of FIG. 2 shown in a first configuration with a side cross-sectional view of a portion of a catheter, and an intraluminal device disposed inside a lumen of the delivery device.

FIG. 4 is a side view shown partially in cross-section of a portion of the delivery system, catheter and intraluminal device of FIG. 2 with the delivery system shown in a second configuration and the intraluminal device disposed partially deployed outside of the lumen of the delivery device.

FIG. 5 is a side view shown partially in cross-section of a portion of the delivery system, catheter and intraluminal device of FIG. 2 with the delivery system shown in an extended configuration and the intraluminal device fully deployed outside the lumen of the delivery device.

FIG. 6 is a side view shown partially in cross-section of a portion of the delivery system, catheter and intraluminal device of FIG. 2 with the delivery system shown in a retracted position and the intraluminal device disposed partially deployed outside of the lumen of the delivery device.

FIGS. 7 and 8 are each a side view shown partially in cross-sectional of a portion of the delivery system, catheter and intraluminal device of FIG. 2 illustrating the delivery system and intraluminal device being retracted proximally within a lumen of the catheter.

FIG. 9 is a side view shown partially in cross-section of a portion of a delivery system according to another embodiment shown in first configuration.

FIG. 10 is a side view shown partially in cross-section of a portion of the delivery system of FIG. 9 shown in a second configuration and an intraluminal device disposed partially deployed outside of the lumen of the delivery device.

FIG. 11 is a side view shown partially in cross-section of a portion of the delivery system of FIG. 10 with the delivery system in a retracted position illustrating the intraluminal device being retracted proximally within a lumen of the delivery system.

FIG. 12 is a is a side view shown partially in cross-section of a portion of a delivery system according to another embodiment.

FIG. 13A is a side view shown partially in cross-section of a portion of a delivery system according to another embodiment shown in first configuration.

FIG. 13B is a side view shown partially in cross-section of a portion of the delivery system of FIG. 13A shown in a second configuration and an intraluminal device partially deployed outside of the lumen of the delivery device.

FIG. 13C is a side view shown partially in cross-section of a portion of the delivery system of FIG. 13A shown in a third configuration and an intraluminal device fully deployed outside of the lumen of the delivery device.

FIG. 13D is a perspective view of a portion of the delivery system of FIG. 13A.

FIG. 14A is a side view shown partially in cross-section of a portion of a delivery system according to another embodiment shown in first configuration.

FIG. 14B is a side view shown partially in cross-section of a portion of the delivery system of FIG. 14A shown in a second configuration and an intraluminal device partially deployed outside of the lumen of the delivery device.

FIG. 14C is a side view shown partially in cross-section of a portion of the delivery system of FIG. 14A shown in a third configuration and an intraluminal device fully deployed outside of the lumen of the delivery device.

FIGS. 15 and 16 are each a flowchart illustrating a method of deploying a medical device according to different embodiments.

FIGS. 17-19 are each a Table illustrating the elongation and change in diameter for different example embodiments of a medical device.

FIG. 20 is a graph illustrating the percentage elongation and corresponding diameter for the example embodiments of a medical device of FIGS. 17-19.

### DETAILED DESCRIPTION

The devices and methods described herein are configured for use in the treatment and/or diagnosis of blood vessels, such as cerebral blood vessels. For example, devices and methods are described herein to treat cerebrovascular pathologies, such as intracranial aneurysms or “brain” aneurysms. In some embodiments, an apparatus and method for treating a site at a cerebral blood vessel includes carrying a medical treatment element (also referred to herein as “medical device”), such as a medical implant or intraluminal device, a flow diverter, or an aneurysm neck occlusion device to a treatment site within a cerebral blood vessel. For example, when the treatment site includes an aneurysm neck, the deployment of a medical device, such as an implant, decreases the blood flow within the aneurysm, permitting the remaining blood in the aneurysm to coagulate and decrease the likelihood that the aneurysm will rupture.

In some embodiments, a delivery system is provided for carrying and delivering a medical device and provides for the recapture and redeployment of the medical device as described in more detail below. Also described herein, in some embodiments, all or some of the components of a delivery system can be included in a kit. For example, a delivery device and a cannula can be provided in a kit.

In some embodiments, a delivery system for a medical device includes a flexible sheath having an interior wall bounding a lumen having a distal end and a flexible shaft having a proximal portion and a distal portion. A retention element and a pushing element are each disposed on the distal portion. The pushing element is spaced proximally from the retention element. The flexible shaft, retention element, and pushing element are disposable within the lumen to collectively define with the interior wall a region sized to contain the medical device. The flexible shaft is movable distally through the lumen between a stowed position in which the retention element is proximal to the distal end of the lumen and a deployed position in which the pushing element at least partially extends distally from the distal end of the lumen. The flexible shaft is movable proximally through the lumen between the deployed position and a partially deployed position in which the retention element is proximate to the distal end of the lumen. The retention element is configured to form with the medical device an interference fit with the flexible sheath when the flexible shaft is urged proximally from to the partially deployed position to a retention position to engage the medical device with the flexible sheath.

In some embodiments, a kit includes a delivery device including a sheath having a distal end and an interior wall bounding a lumen and a flexible shaft having a proximal portion and a distal portion. The flexible shaft includes a retention element and a pushing element disposed on the distal portion and is disposable within the lumen of the sheath. The kit also includes a medical device mountable on the flexible shaft and can be constrained within the sheath in a radially compressed configuration. The flexible shaft is movable distally through the lumen between a stowed position in which the retention element is proximal to the distal end of the lumen and a deployed position in which the pushing element at least partially extends distally from the distal end of the lumen. The flexible shaft is movable proximally through the lumen between the deployed position and a partially deployed position in which the retention element is proximate to the distal end of the lumen. The retention element is configured to form with the medical device an interference fit with the sheath when the flexible shaft is urged proximally from the partially deployed position to a retention position to engage the medical device with the sheath.

In some embodiments, a method is provided for disposing in a blood vessel at a treatment site adjacent an aneurysm a self-expanding medical device using a sheath having a distal end and an interior wall defining an internal lumen and a flexible shaft having a proximal portion and a distal portion. The flexible shaft includes a retention element disposed at the distal portion and is movable through the lumen. The medical device is mounted on the flexible shaft and constrained within the sheath in a radially compressed configuration. The method includes positioning the distal end of the sheath near the treatment site. The sheath is moved proximally relative to the flexible shaft to a partially deployed position in which the retention element is sufficient distance distal from the distal end of the sheath to allow a portion of the medical device to self-expand beyond the distal end of the sheath to engage an inner wall of the blood vessel near the treatment site. The flexible shaft is partially retracted so as to frictionally engage the medical device between the retention element and the sheath.

It is noted that, as used in this written description and the appended claims, the singular forms “a,” “an” and “the” include plural referents unless the context clearly dictates otherwise. Thus, for example, the term “a lumen” is intended to mean a single lumen or a combination of lumens. Furthermore, the words “proximal” and “distal” refer to direction closer to and away from, respectively, an operator (e.g., surgeon, physician, nurse, technician, etc.) who would insert a medical device into the patient, with the tip-end (i.e., distal end) of the device inserted inside a patient's body. Thus, for example, the end inserted inside a patient's body would be the distal end of the medical device, while the end outside a patient's body would be the proximal end of the medical device.

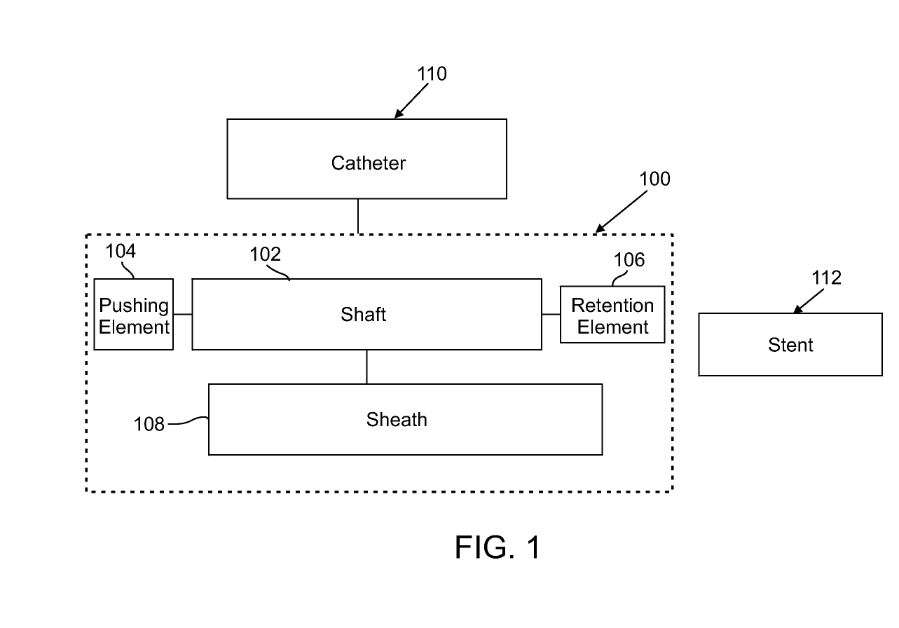

FIG. 1 is a schematic illustration of an embodiment of a delivery system (also referred to herein as “delivery device”). A delivery system 100 can include an elongate flexible shaft 102, a pushing element 104 and a retention element 106. The pushing element 104 and/or the retention element 106 can be coupled to, or formed monolithically with (or integrally) the flexible shaft 102. The delivery system 100 also includes a sheath 108 that has an interior wall defining an interior lumen (not shown in FIG. 1) that can movably receive the elongate flexible shaft 102, the pushing element 104 and the retention element 106 therein.

The flexible shaft 102 can be a solid component (e.g., a solid rod), or can define one or more lumens (not shown in FIG. 1). For example, in some embodiments, the flexible shaft 102 can define a lumen through which a guidewire can be received. The flexible shaft 102 can be coupled to a handle (not shown in FIG. 1) at a proximal end portion of the flexible shaft 102 to be used by a medical practitioner to help maneuver the delivery device 100 within a patient. The flexible shaft 102 can be formed such that it is sufficiently flexible to traverse tortuous blood vessels while having sufficient firmness to be maneuverable within the blood vessel of a patient. The flexible shaft 102 can be formed with biocompatible materials used in medical devices. For example, the flexible shaft 102 can be formed with various biocompatible metals or plastics, such as, for example, various polymers, polyurethane, polyester, polyethylene or silicon. As described in more detail below, the flexible shaft 102 has an outer perimeter (or outer diameter) that is smaller than an inner perimeter (or inner diameter) of a lumen of the flexible sheath 108 such that the flexible shaft 102 can be movably disposed therethrough.

The pushing element 104 can be coupled to the flexible shaft 102 and is disposed at a distal portion of the shaft 102 and proximal of the retention element 106, as shown and described in more detail below with reference to specific embodiments. The pushing element 104 can be coupled to the flexible shaft 102 with, for example, an adhesive or a friction fit, or other suitable coupling method. For example, in some embodiments, the pushing element 104 can define a lumen therethrough and the flexible shaft 102 can be slidably received through the lumen of the pushing element 106 and coupled thereto. In some embodiments, the pushing element 104 can be coupled to a portion of an exterior surface of the flexible shaft 102. Thus, the pushing element 104 can be configured to surround entirely the flexible shaft 102, or can be coupled to a portion of the exterior surface of the flexible shaft 102. Alternatively, the pushing element 104 can be formed monolithically with the flexible shaft 102.

The pushing element 104 can be formed with one or more flexible, semi-rigid and/or rigid biocompatible materials and is sized to enable the pushing element 104 to slidably move within the lumen of the sheath 108. Thus, an outer perimeter (or outer diameter) of the pushing element 104 is smaller than an inner perimeter (or inner diameter) of the lumen of the sheath 108.

The retention element 106 can be coupled to the flexible shaft 102 and is disposed at a distal portion of the flexible shaft 102, as shown in more detail below with reference to specific embodiments. The retention element 106 can be coupled to the flexible shaft 102 with, for example, an adhesive or a friction fit, or other suitable coupling method. For example, in some embodiments, the retention element 106 can define a lumen therethrough and the flexible shaft 102 can be slidably received through the lumen of the retention element 106 and coupled thereto. Alternatively, the retention element 106 can be formed monolithically with the flexible shaft 102. In some embodiments, the retention element 106 can be disposed at a distal end of the shaft 102. In some embodiments, the retention element 106 defines an opening, rather than a lumen extending through the retention element 106. In such an embodiment, a distal end of the flexible shaft 102 can be inserted into the opening and coupled thereto, for example with an adhesive.

The retention element 106 can be formed with a resilient material such as, for example, an elastic material, such as rubber, urethane, polypropylene, polycarbonate, and/or a foam material and/or a combination thereof. The retention element 106 can have a hardness rating, for example, in a range of about 30 on a Shore A scale to about 90 on a shore D scale. In some embodiments, the retention element 106 can have a hardness rating in any sub-range within this range. For example, in some embodiments, the retention element 106 can have a hardness rating in the range of about 30-90 on a shore A scale. In some embodiments, the retention element 106 can have a hardness rating in a range of about 30-90 on a shore D scale. In some embodiments, the retention element 106 can have a hardness rating in a range of less than about 90, 85, 80, 75, 70, 65, 60, 55, 50, 45, 40 or 35 on the Shore A scale. In some embodiments, the retention element 106 can have a hardness rating of greater than about 30, 35, 40, 45, 50, 55, 60, 65, 70, 75, 80, or 85 on the Shore D scale. The retention element 106 is sized to enable it to be slidably moved within the lumen of the sheath 108. The retention element 106 is also sized to enable the retention element 106 to slidably move within an interior of an expanded medical device, such as an intraluminal device or a stent, as described in more detail below. Thus, similar to the pushing element 104, an outer perimeter (or outer diameter) of the retention element 106 is smaller than an inner perimeter (or inner diameter) of the lumen of the sheath 108. In some embodiments, the retention element 106 can have a variable outer and/or inner perimeter (or diameter). For example, the retention element 106 can be tapered, cone-shaped, or bullet-shaped. Such an embodiment may be desirable to provide a narrower distal end or lead-in portion of the retention element to improve maneuverability, for example, within a lumen or blood vessel,

As mentioned above, the flexible sheath 108 has an interior wall that defines a lumen that extends from a proximal end to a distal end of the flexible sheath 108. The flexible shaft 102, the pushing element 104 and the retention element 106 are each sized and configured to be movably disposable within the lumen of the flexible sheath 108. The flexible sheath 108 can be formed with a material such that it is sufficiently flexible to traverse tortuous blood vessels while having sufficient firmness to be maneuverable within the blood vessel of a patient. In some embodiments, the flexible sheath 108 is formed with a material such that it has a hardness rating in a range of about 20-90 on a Shore D scale. In some embodiments, the flexible sheath 108 has a hardness rating greater than about 30 on the Shore D scale. In some embodiments, the flexible sheath 108 has a hardness rating of greater than about 45 on the Shore D scale. In some embodiments, the flexible sheath 108 has a hardness rating of about 35 on a Shore D scale. The flexible sheath 108 can be sized and shaped to fit through a blood vessel, such as a cerebral blood vessel. For example, in some embodiments, the flexible sheath 108 has an outer diameter in a range of about 0.5 mm (0.002 inches) to about 3.0 mm (0.07 inches). In some embodiments, the flexible sheath 108 can have an outer diameter in any sub-range within this range. For example, in some embodiments, the flexible sheath 108 has an outer diameter in a range of about 1.0 mm (0.04 inches) to about 3.0 mm (0.07 inches). In some embodiments, the flexible sheath 108 can have an outer diameter in a range of about 0.5 mm (0.002 inches) to about 2.0 mm (0.08 inches). In some embodiments, the flexible sheath 108 has an outer diameter of about 2.0 mm (0.08 inches). In some embodiments, the flexible sheath 108 has an outer diameter of about 0.7 mm (0.027 inches).

In some embodiments, the flexible sheath 108 can include a stiffening member (not shown in FIG. 1), such as a helical coil. For example, a helical coil can be coupled to or embedded within the material of the flexible sheath 108 to provide strengthening properties while remaining flexible. In some embodiments, the flexible shaft 102 can include a stiffening member as described herein. Other types of stiffeners can alternatively be used, such as, for example, composite stiffeners which are intertwined within the material of the flexible sheath 108 and/or flexible shaft 102, or otherwise coupled thereto, and/or any other element that can change the stiffness and/or elasticity coefficients of the flexible sheath 108 and/or the flexible shaft 102. In some embodiments, a stiffening member can be coupled to or disposed at different locations along a length of the flexible sheath 108 and/or flexible shaft 102, for example, to vary the flexibility along the length of the flexible sheath 108 and/or flexible shaft 102.

In some embodiments, delivery device 100 is used in conjunction with a guide catheter 110. The guide catheter 110 can be a variety of known guide catheters used for treatment within a blood vessel. Thus, in such an embodiment, the guide catheter 110 can be sized and shaped to fit through a blood vessel, such as a cerebral blood vessel. The guide catheter 110 can have an interior wall that defines a lumen (not shown in FIG. 1) through which the delivery device 100 can be movably disposed. The interior wall of the catheter 110 can be smooth or have a roughened surface to provide friction as a medical device is passed through the lumen of the catheter 110. In such an embodiment in which a catheter 110 is used, the flexible sheath 108 can be sized and shaped to be movably disposable within the lumen of the guide catheter 110. For example, the flexible sheath 108 has an outer perimeter (or outer diameter) that is less than an inner perimeter (or inner diameter) of the lumen of the guide catheter 110.

The delivery device 100 can be used, for example, to deliver and deploy a medical device 112, such as, for example, an intraluminal device, such as, for example, a medical implant or stent, a flow diverter, or an aneurysm neck occlusion device, to a treatment site within a cerebral blood vessel. The medical device 112 can be variety of different configurations. For example, the medical device 112 can be a self-expanding tubular device, a coiled device, a braided, woven or knitted device, a mesh device and/or any other device that is configured to be placed within a blood vessel, such as on, or in proximity to, for example, an aneurysm neck, an occlusion and/or constriction in a blood vessel, an arteriovenous malformation and/or any other cerebrovascular pathology. The medical device 112 can also be a laser cut, a photochemically etched and/or electropolished device.

The medical device 112 can be formed with various biocompatible materials, such as, for example, various biocompatible metals and or plastics or polymers. For example materials, such as, stainless steel, tantalum, super elastic Nitinol, cobalt base alloy, platinum, polymers or any other suitable metal or metal combination, plastic or plastic combination can be used. The medical device 112 can also be coated with bio-compatible coatings. It is also possible to use combination of several different materials or coatings to achieve radio-opacity. The medical device 112 can also be biodegradable or dissolvable within a blood vessel. The medical device 112 can also be formed a shape memory alloy (SMA), and/or a deformable material such that medical device 112 is self-expanding. For example, the medical device 112 can be a self expanding braided tubular device that is collapsible and capable of self-expanding to an expanded shape. Examples of medical devices that can be delivered with a delivery system as described herein are also described in U.S. Patent Pub. No. 2008/0039933, the entire disclosure of which is hereby incorporated herein by reference.

In some embodiments, in which the medical device 112 is an expanding braided tubular device, the medical device 112 can have a relaxed configuration in which the medical device 112 has a length and an outer diameter (or outer perimeter), and an elongated configuration in which the medical device 112 has a length that is greater than the length of the medical device 112 when in the relaxed configuration and an outer diameter (or outer perimeter) that is smaller than the outer diameter (or outer perimeter) when in the relaxed configuration. For example, to move the medical device 112 from the relaxed configuration to the elongated configuration, the ends of the medical device 112 can be pulled in opposite directions to elongate or stretch the medical device 112. When the medical device 112 is in the relaxed configuration, a flexibility of the medical device 112 will be greater than a flexibility of the medical device 112 when in the elongated configuration. Thus, to achieve greater flexibility of the delivery system 100 when the medical device 112 is coupled thereto, it may be desirable for the medical device 112 to be in its relaxed configuration. The lumen of the flexible sheath 108 can be sized to receive the medical device 112 therein when in the relaxed configuration. Such a configuration of the delivery device 100 can improve the flexibility and, therefore, the maneuverability of the distal end portion of the delivery device 100 through a tortuous vessel (e.g., cerebral blood vessel).

The medical device 112 can also have a semi-relaxed or partially relaxed configuration. For example, when the medical device 112 is in a partially relaxed configuration it can have a length that is greater than when in the relaxed configuration and that is smaller than the length when in the elongated configuration. Similarly, when the medical device 112 is in a partially relaxed configuration it can have an outer diameter or outer perimeter that is slightly smaller than the outer diameter or outer perimeter of the medical device 112 when in the relaxed configuration and larger than an outer diameter or outer perimeter of the medical device 112 when in the elongated configuration. It may be desirable to position the medical device 112 within the lumen of the flexible sheath 108 in a partially relaxed configuration. In such an embodiment, the delivery device 100 can still have improved flexibility, while at the same time a reduced diameter of the medical device 112 and, therefore, an overall reduced diameter of the delivery device 100 can be achieved.

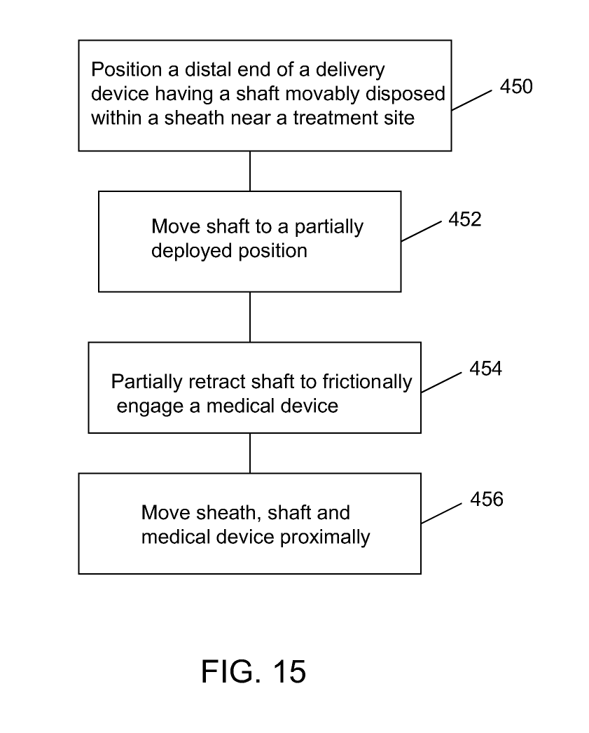

FIGS. 15-17 are each a table illustrating examples of the amount of elongation and corresponding change of diameter that can occur with various example sizes of a medical device 112. FIG. 18 is a graphical illustration of the percentage elongation versus change in diameter for the three example medical devices of Tables 1-3. In each table, the first column is the outer diameter of the medical device and the second column is the corresponding percentage elongation. For example, FIG. 15 illustrates the elongation and change in diameter for an example medical device 112 having an outer diameter of 3.7 mm and a length of between, for example 20-30 mm, when in a fully relaxed configuration. Such a medical device 112 can be used, for example, in an artery that is slightly smaller than the medical device 112 in the relaxed configuration, such as an artery having a diameter of about 3.5 mm As the medical device 112 is elongated, the diameter of the medical device 112 is correspondingly reduced. For example, as the medical device 112 is elongated to 116% of its original length, the corresponding diameter of the medical device is reduced from 3.7 mm to 3.4 mm. In this example, the medical device 112 can be elongated 175% and have a diameter of 1.016 mm when placed in the lumen of the flexible sheath 108 of the delivery device 100. The fully elongated length of the medical device 112 in this example is 0.7 mm (177% elongation). The partially relaxed configuration can include any configuration in which the medical device is between the fully relaxed configuration and the fully elongated configuration.

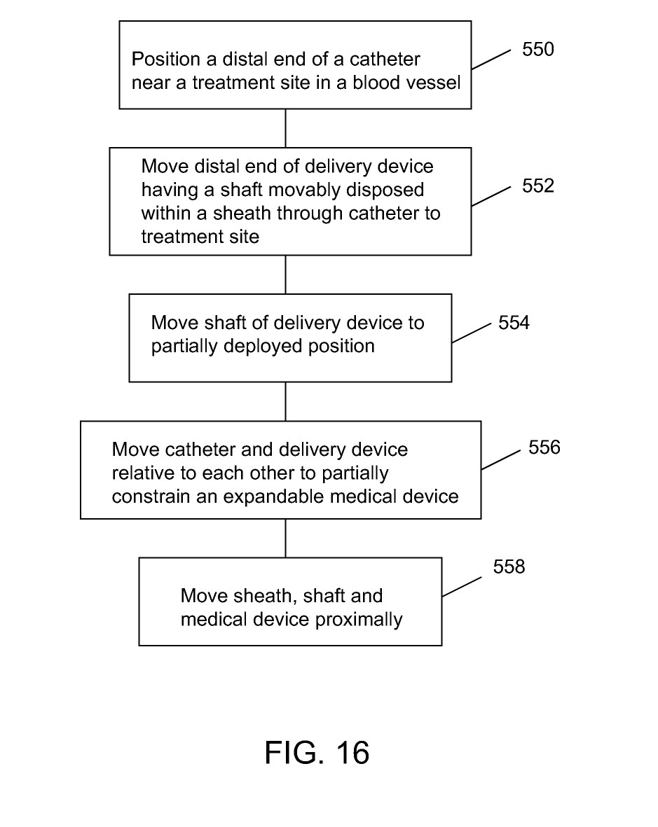

FIG. 16 illustrates the elongation and change in diameter for an example medical device 112 having an outer diameter of 4.6 mm and a length of between, for example 20-30 mm, when in a fully relaxed configuration. Such a medical device 112 can be used, for example, in an artery that is slightly smaller than the medical device 112 in the relaxed configuration, such as an artery having a diameter of about 4.4 mm. As the medical device 112 is elongated, the diameter of the medical device 112 is correspondingly reduced as illustrated in Table 2 of FIG. 16. For example, as the medical device 112 is elongated to 122% of its original length, the corresponding diameter of the medical device is reduced from 4.6 mm to 4.24 mm. In this example, the medical device 112 can be elongated 203% and have a diameter of 1.016 mm when placed in the lumen of the flexible sheath 108 of the delivery device 100. The fully elongated length of the medical device 112 in this example is 1.0 mm (203% elongation). The partially relaxed configuration can include any configuration in which the medical device is between the fully relaxed configuration and the fully elongated configuration.

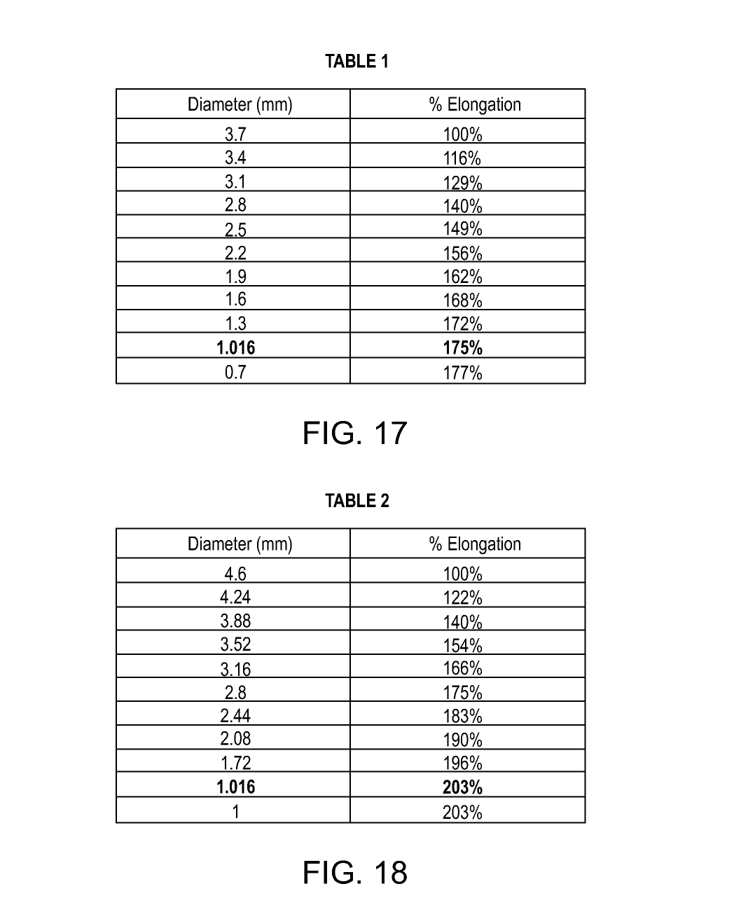

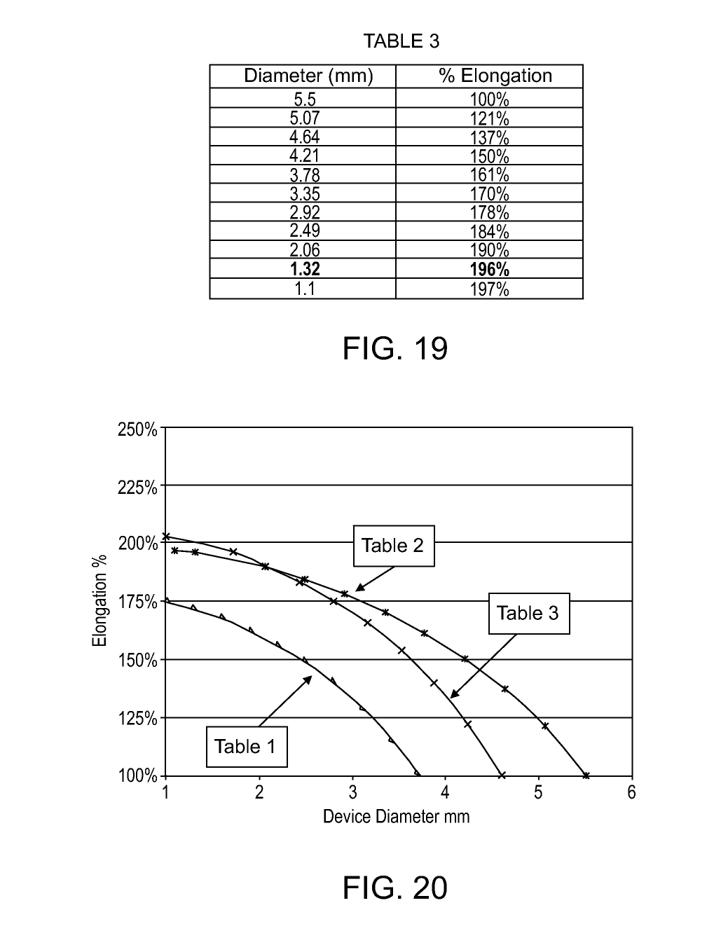

FIG. 17 illustrates the elongation and change in diameter for an example medical device 112 having an outer diameter of 5.5 mm and a length of between, for example 20-30 mm, when in a fully relaxed configuration. Such a medical device 112 can be used, for example, in an artery that is slightly smaller than the medical device 112 in the relaxed configuration, such as an artery having a diameter of about 5.3 mm. As the medical device 112 is elongated, the diameter of the medical device 112 is correspondingly reduced as illustrated in Table 3 of FIG. 17. For example, as the medical device 112 is elongated to 137% of its original length, the corresponding diameter of the medical device is reduced from 5.5 mm to 4.64 mm. In this example, the medical device 112 can be elongated 196% and have a diameter of 1.32 mm when placed in the lumen of the flexible sheath 108 of the delivery device 100. The fully elongated length of the medical device 112 in this example is 1.1 mm (197% elongation). The partially relaxed configuration can include any configuration in which the medical device is between the fully relaxed configuration and the fully elongated configuration.

To deliver the medical device 112 to a treatment site, the medical device 112 can be placed inside the lumen of the flexible sheath 108 (near a distal end of the flexible sheath 108), such that the medical device 112 surrounds the flexible shaft 102 and is disposed at a location between the pushing element 104 and the retention element 106. Thus, the pushing element 104 is disposed proximally of a proximal end of the medical device 112 and the retention element 106 is disposed distally of a distal end of the medical device 112.

In this example method, the medical device 112 is a collapsible and self-expanding intraluminal device. For example, to insert the medical device 112 within the delivery device 100, the flexible shaft 102 can be disposed in the lumen of the flexible sheath 108 and advanced toward the distal end of the flexible sheath 108 until the pushing element 104 is disposed outside of the distal end of the lumen of the flexible sheath 108 (e.g., about 30 mm beyond the distal end of the sheath 108). The medical device 112 can then be disposed over the flexible shaft 102 and positioned between the pushing element 104 and the retention element 106. The flexible sheath 108 can then be moved distally over the flexible shaft 102 and the medical device 112, while slightly compressing or deforming a proximal end portion of the medical device 112 to introduce it within the lumen of the flexible sheath 108. As the flexible sheath 108 is moved over the medical device 112 it will radially compress it within the lumen of the flexible sheath 108. In some embodiments, it may be desirable to position the medical device 112 within the lumen of the flexible sheath 108 at a spaced distance (e.g., about 1 cm) from a distal end of the flexible sheath 108. Positioning the medical device 112 away from the distal end can help provide more flexibility at the distal end of the flexible shaft 108.

When the medical device 112 is disposed within the lumen of the flexible sheath 108 at a desired location, it is in a stored or stowed configuration in which the medical device 112 has an outer perimeter (or outer diameter) that is smaller than the inner perimeter (or inner diameter) of the lumen of the flexible sheath 108. Thus, the medical device 112 is in a radially compressed configuration within the lumen of the flexible sheath 108. When the medical device 112 is deployed at a treatment site it can self-expand to a deployed or expanded configuration in which the medical device 112 has an outer perimeter (or outer diameter) that is larger than its stored outer perimeter (or outer diameter). In some embodiments, the delivery device 100 can include the pushing element 104, retention element 106 and the medical device 112 pre-positioned within the sheath 108 in a stowed or stored position.

With the medical device 112, the pushing element 104 and the retention element 106 disposed within the lumen of the flexible sheath 108, the delivery device 100 can be inserted through a lumen of a guide catheter (e.g., catheter 110) that has been inserted into the blood vessel and positioned with its distal end at or near a desired treatment site. For example, the guide catheter 110 can be introduced percutaneously into a large blood vessel, such as the femoral artery or vein near the groin, and maneuvered to the desired treatment location. In some embodiments where at least a portion of the delivery device is radiopaque, an imaging device such as a fluoroscope can be used to help guide the delivery device to a desired treatment site. For example, in some embodiments, a portion of the distal end portion of the flexible sheath 108 can be radiopaque and/or a portion of a distal end portion of the flexible shaft 102 can be radiopaque and/or a portion of a distal end portion of a guide catheter 110 can be radiopaque.

When the distal end of the delivery device 100 is at a desired location (e.g., an aneurysm neck or another cerebrovascular pathology region within a cerebral blood vessel), the flexible shaft 102 can be moved distally relative to the flexible sheath 108 such that the retention element 106 is moved distally outside of the distal end of the lumen of the flexible sheath 108 and within the blood vessel. The pushing element 104 can contact and exert a force on the proximal end of the medical device 112, moving the medical device 112 distally. The medical device 112 can be moved distally until at least a portion of the medical device 112 is extended or deployed outside of the lumen of the flexible sheath 108 and allowed to at least partially expand within the blood vessel. The medical practitioner can continue to move the flexible shaft 102 distally until the entire medical device 112 is deployed or implanted at the treatment site. Alternatively, before the medical device 112 is fully deployed, the medical practitioner can recapture the medical device 112 and move the distal end of the delivery device 100 (and the guide catheter 110) to a different location within the blood vessel.

To recapture the medical device 112 after it has been partially deployed, the flexible shaft 102 is moved proximally to a retention position in which the retention element 106 engages an interior of the medical device 112 and the distal end of the flexible sheath 108. As the flexible shaft 102 continues to be moved proximally, the retention element 106 and the medical device 112 together form an interference fit with the flexible sheath 108. In other words, the medical device can be grasped or pinched between the flexible sheath 108 and the retention element 106. The resilient material of the retention element 106 enables the retention element 106 to be maneuvered within the blood vessel and also helps form the interference fit with the flexible sheath 108.

The delivery device 100 can then be urged or pulled proximally relative to the guide catheter 110, such that the medical device 112 is withdrawn proximally into the lumen of the guide catheter 110. As the medical device 112 is pulled into the lumen of the guide catheter 110, the medical device 112 deforms or collapses to fit within the guide catheter 110. With the medical device 112 withdrawn within the guide catheter 110, the guide catheter and delivery device 100 can be repositioned at a different location within the blood vessel to redeploy the medical device 112, or can alternatively be removed from the blood vessel.

In the above example method, the flexible shaft 102 was described as being moved relative to the flexible sheath 108. Alternatively, the flexible sheath 108 can be moved proximally relative to the flexible shaft 102. In this example, as the flexible sheath 108 is moved proximally, the retention element 106 becomes disposed outside of the distal end of the lumen of the flexible sheath 108 and within the blood vessel and at least a portion of the medical device 112 becomes disposed within the blood vessel as the radial constraint of the flexible sheath 108 is removed. The pushing element 104 can prevent the medical device 112 from moving proximally as the flexible sheath 108 is moved proximally. The flexible sheath 108 can be moved proximally until the entire medical device 112 is deployed or implanted at the treatment site, or as described above, the medical device 112 can be recaptured and moved to a different location within the blood vessel as described above.

In another alternative, the delivery device 100 can include a guidewire lumen (not show in FIG. 1) such that the delivery device 100 can be inserted directly into the blood vessel and directed to the treatment site with a guidewire, rather that using a guide catheter. In such an alternative, the deployment of the medical device 112 can be effected in the same manner as described above. More details related to the recapturing process and various methods of use of the delivery device 100 are described below with reference to specific embodiments.

Having described above various general examples, examples of specific embodiments are described below. These embodiments are only example, and many other configurations and uses of the devices described herein are contemplated.

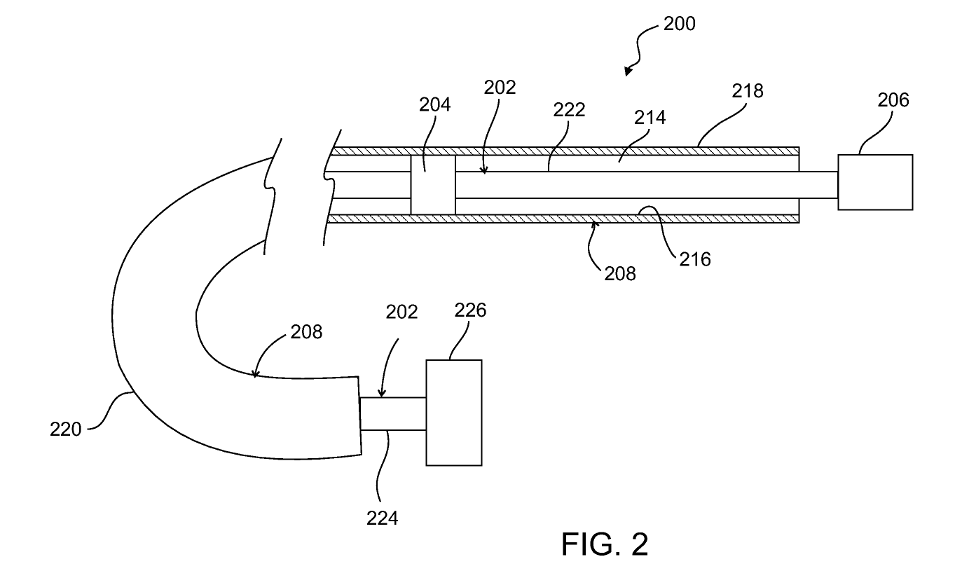

FIGS. 2-8 illustrate a delivery system according to an embodiment. As shown in FIG. 2, a delivery system 200 includes a flexible sheath 208, a flexible shaft 202, a pushing element 204 and a retention element 206. The various components of the delivery device 200 can be formed and configured in the same manner as described above for delivery device 100, and therefore, will not be described in detail below with reference to this embodiment. The flexible sheath 208 has an interior wall 216 defining a lumen 214 that extends between a distal end portion 218 and a proximal end portion 220 of the flexible sheath 208. The flexible shaft 202 includes a distal end portion 222 and a proximal end portion 224. The proximal end portion 224 can optionally be coupled to a handle 226.

The retention element 206 is coupled to the distal end portion 222 of the flexible shaft 202 and is disposed at the distal end of the shaft 202. It should be understood, however, that the retention element 206 can alternatively be disposed at a spaced distance from the distal end of the flexible shaft 202. The pushing element 204 is also coupled to the distal end portion 222 of the flexible shaft 202 and is disposed proximally from the retention element 206.

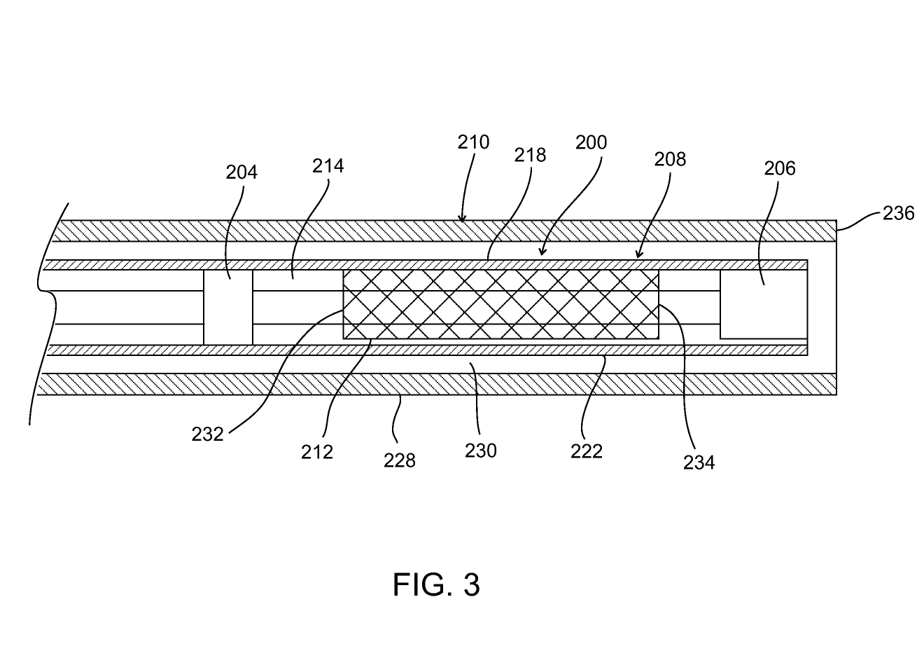

In this embodiment, the delivery device 200 is shown coupled to, and used in conjunction with, a guide catheter 210. FIG. 3 illustrates a distal end portion 228 of the guide catheter 210, and a distal portion of the delivery device 200 disposed within a lumen 230 of the guide catheter 210 in a stowed position. A self-expanding braided medical device 212 is shown disposed within the lumen 214 of the flexible sheath 208 at the distal end portion 218 of the flexible sheath 208. The medical device 212 is tubular shaped and includes a proximal end 232 and a distal end 234.

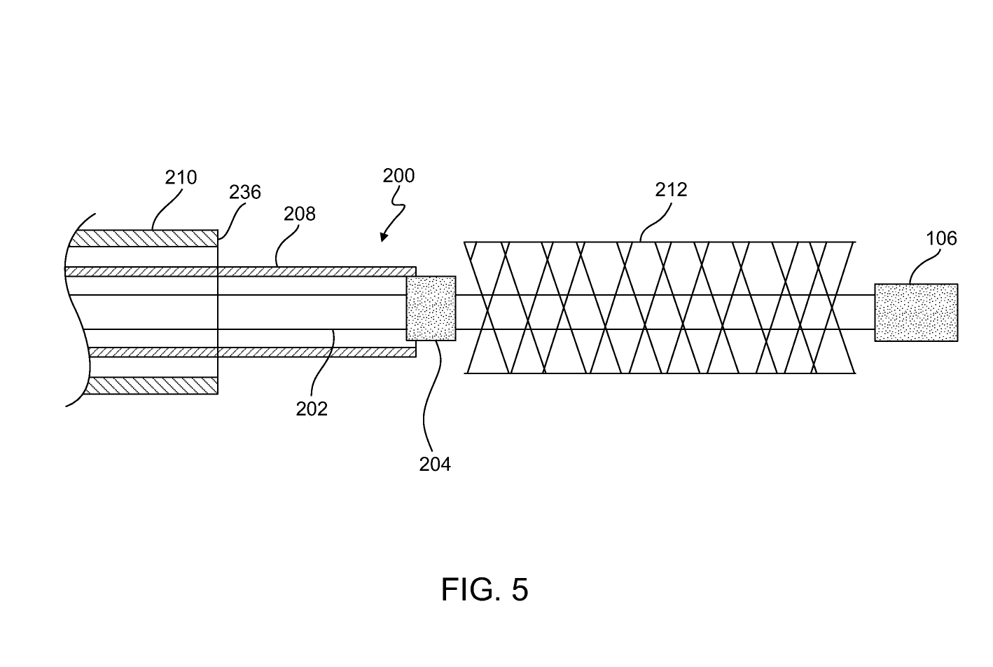

In use, to deliver and deploy the medical device 212 to a treatment site, such as, for example, on or in proximity to, an aneurysm neck and/or any other cerebrovascular pathology within a cerebral blood vessel, the medical device 212, the pushing element 204 and the retention element 206 are placed within the lumen 214 of the flexible sheath 208. The medical device 212 is positioned such that the medical device 212 surrounds the flexible shaft 202 at a location between the pushing element 204 and the retention element 206 as shown in FIG. 2. Thus, the pushing element 204 is disposed proximally of a proximal end 232 of the medical device 212 and the retention element 206 is disposed distally of a distal end 234 of the medical device 212. When the medical device 212 is disposed within the lumen 214 of the flexible sheath 208, it is in a stored or stowed configuration in which the medical device 212 has an outer perimeter (or outer diameter) that is smaller than the inner perimeter (or inner diameter) of the lumen 214 of the flexible sheath 208. When the medical device 212 is deployed at a treatment site it can self-expand to a deployed configuration in which the medical device 212 has an outer perimeter (or outer diameter) that is larger than its stored outer perimeter (or outer diameter) (see e.g., FIG. 5).

With the medical device 212, the pushing element 204 and the retention element 206 disposed within the lumen 214 of the flexible sheath 208, the delivery device 200 can be inserted through the lumen 230 of the guide catheter 210. The guide catheter 210 can be positioned within the blood vessel with a distal end 236 of the guide catheter disposed at a desired treatment site prior to insertion of the delivery device 200, or the guide catheter 210 with the delivery device 200 disposed therein can together be inserted into the blood vessel and maneuvered to the desired treatment site. For example, the guide catheter 210 (or guide catheter 210 and delivery device 200) can be introduced percutaneously into a large blood vessel, such as the femoral artery or vein near the groin, and maneuvered to the desired treatment location.

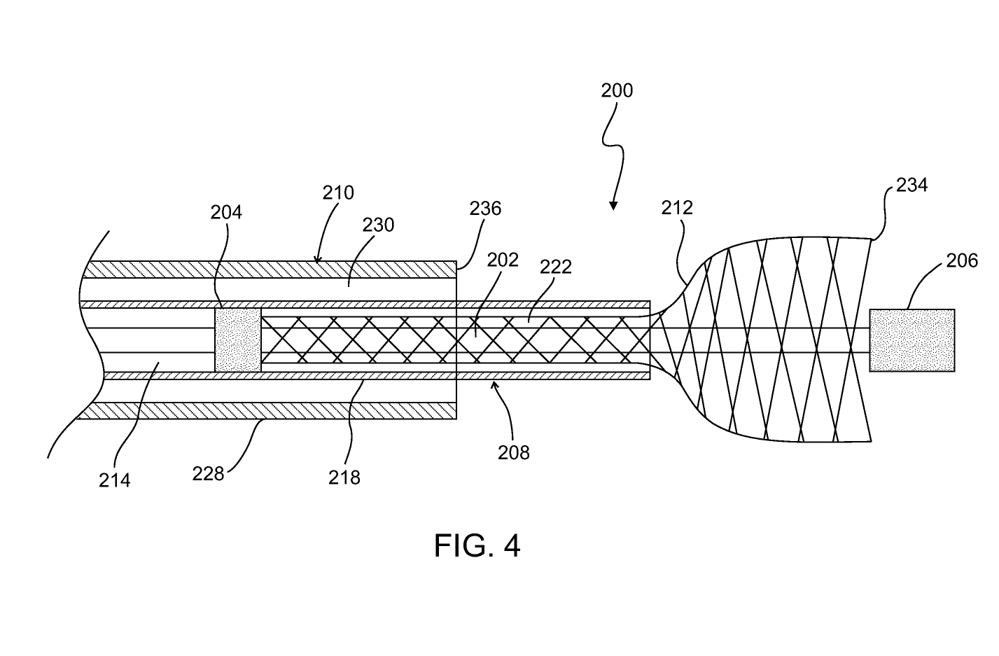

With the distal end 236 of the guide catheter 210 disposed at the treatment site, the delivery device 200 can be moved or urged distally relative to the guide catheter 210 such that the distal end portion 218 of the flexible sheath 208 is moved at least partially outside of the lumen 230 at the distal end 236 of the guide catheter 210. Simultaneously or sequentially, the flexible shaft 202 can be moved distally relative to the flexible sheath 208 such that the retention element 206 is moved distally outside of the distal end of the lumen 214 of the flexible sheath 208 and within the blood vessel in a partially deployed position, and the pushing element 204 contacts or engages the proximal end 232 of the medical device 212, as shown in FIG. 4. As the flexible shaft 202 is moved distally, the pushing element 204 exerts a force on the proximal end 232 of the medical device 212 moving the medical device 212 distally until at least a portion of the medical device 212 is extended or deployed outside of the lumen 214 of the flexible sheath 208 and within the blood vessel. The portion of the medical device 212 deployed within the blood vessel can expand as it is removed from the radial compression of the flexible sheath 208.

The medical practitioner can continue to move the flexible shaft 202 distally to a deployed position until the entire medical device 212 is deployed or implanted at the treatment site, as shown in FIG. 5. In this example, after the medical device 212 has been fully deployed within the blood vessel, the delivery device 200 (and guide catheter 210) can be removed. For example, the flexible shaft 202 can be pulled proximally such that the retention element 206 is pulled proximally through a lumen of the medical device 212 and into the lumen 214 of the flexible sheath 208. Simultaneously or sequentially, the guide catheter 210 and delivery device 200 can both be moved proximally for removal from the blood vessel.

As mentioned above, when the medical device 212 is deployed at a treatment site it can self-expand to a deployed or expanded configuration in which the medical device 212 has an outer perimeter (or outer diameter) that is larger than its stored outer perimeter (or outer diameter). Correspondingly, an inner perimeter or inner diameter of the medical device 212 is larger when the medical device 212 is in its deployed or expanded configuration than when in a stowed or collapsed configuration, such that the retention element 212 can fit through the expanded lumen of the medical device 212 for removal.

Alternatively, before the medical device 212 is fully deployed as described above, the medical practitioner can recapture the medical device 212 and move the distal end of the delivery device 200 (and the distal end of the guide catheter 210) to a different location within the blood vessel, or remove them completely from the blood vessel.

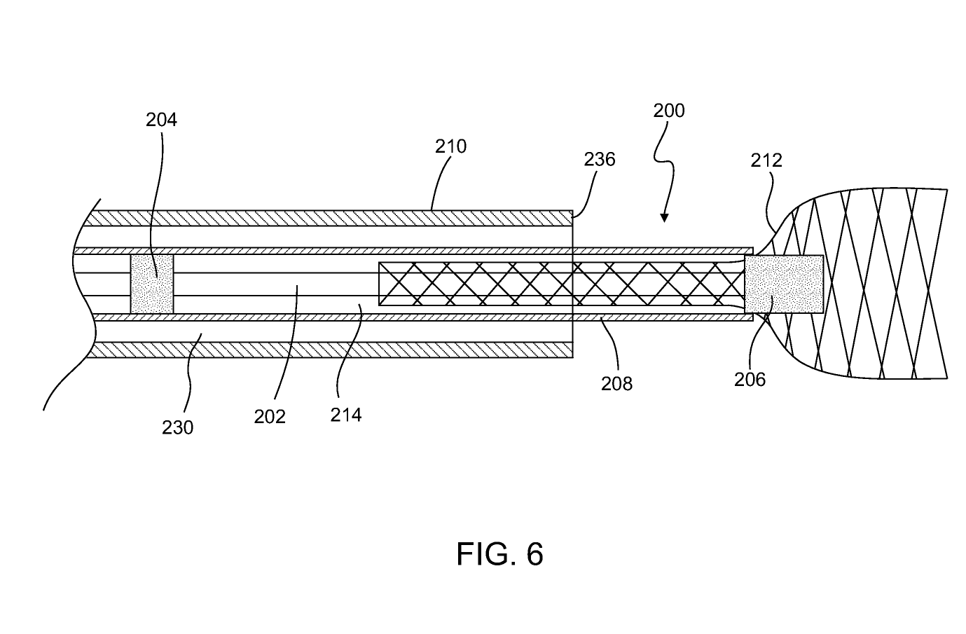

FIGS. 6-8 illustrate the recapture of the medical device 212 to move the medical device 212 to a different location within the blood vessel. With the medical device 212 partially deployed as shown in FIG. 4, the medical practitioner can move the flexible shaft 202 proximally relative to the flexible sheath 208 such that the retention element 206 is pulled or moved proximally through an interior of the deployed or expanded portion of the medical device 212 to a retention position, as shown in FIG. 6. In the retention position, at least a portion of the retention element 206 engages the medical device 212 and the interior wall 216 of the flexible sheath 208. As the flexible shaft 202 continues to be moved proximally, the retention element 206 and the medical device 212 together form an interference fit with the flexible sheath 208. The resilient material of the retention element 206 enables the retention element 206 to be maneuvered within the blood vessel and also helps form the interference fit with the flexible sheath 208.

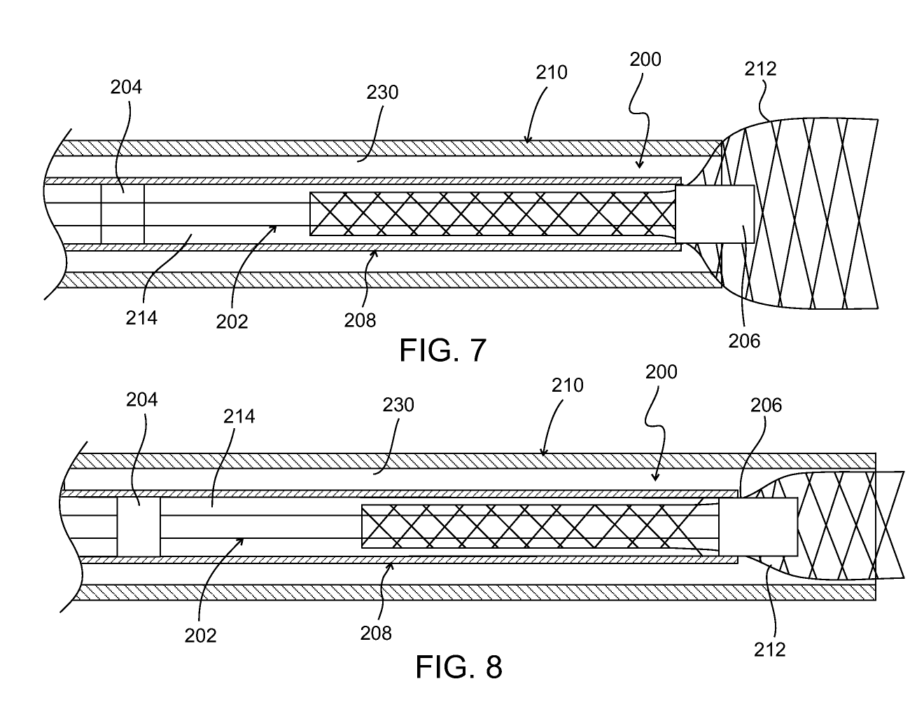

The delivery device 200 can then be urged or pulled proximally relative to the guide catheter 210, such that the medical device 212 is withdrawn or retracted proximally into the lumen 230 of the guide catheter 210, as shown in FIG. 7. FIG. 8 illustrates the further withdrawal of the delivery device 200 and medical device 212 within the guide catheter 210. As the medical device 212 is pulled into the lumen 230 of the guide catheter 210, the portion of the medical device 212 disposed distally of the retention element 206 deforms or collapses to fit within the lumen 230. With the medical device 212 withdrawn within the guide catheter 210, the guide catheter (and delivery device 200 disposed therein) can be repositioned at a different location within the blood vessel to redeploy the medical device 212, or can alternatively be removed from the blood vessel.

In the above example, the flexible shaft 202 was described as being moved relative to the flexible sheath 208 and the delivery device 200 was described as being moved relative to the catheter 210. Alternatively, the flexible sheath 208 can be moved proximally relative to the flexible shaft 202 and the catheter 210 can be moved distally relative to the delivery device 200. In this example, as the flexible sheath 208 is moved proximally, the retention element 206 becomes disposed outside of the distal end of the lumen 214 of the flexible sheath 208 and within the blood vessel and at least a portion of the medical device 212 becomes disposed within the blood vessel as the radial constraint of the flexible sheath 208 is removed. The pushing element 204 can prevent the medical device 212 from moving proximally as the flexible sheath 208 is moved proximally. The flexible sheath 208 can be moved proximally until the entire medical device 212 is deployed or implanted at the treatment site, or as described above, the medical device 212 can be recaptured and moved to a different location within the blood vessel as described above.

In this alternative example, to recapture the medical device 212, the flexible shaft 202 is moved proximally relative to the flexible sheath 208 to the retention position, in the same manner as described above. However, after the flexible shaft 202 is in the retention position, the catheter 210 can be moved distally relative to the flexible sheath 208 until the medical device 212 is constrained within the lumen 230 of the catheter 210.

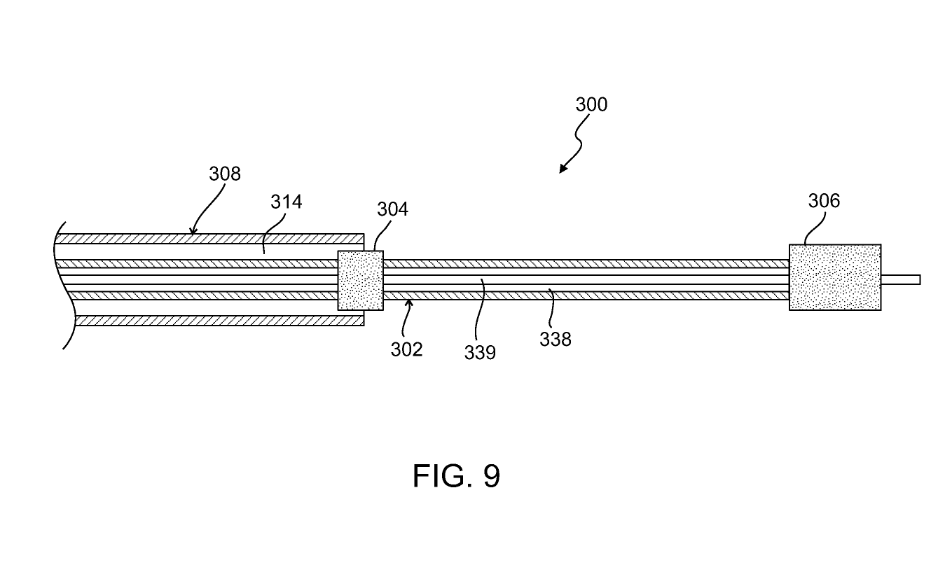

FIGS. 9-11 illustrate a delivery system according to another embodiment. The delivery system 300 includes a flexible sheath 308, a flexible shaft 302, a retention element 306 and a pushing element 304. The flexible sheath 308 defines a lumen 314 (see FIG. 9) that extends between a distal end portion 318 and a proximal end portion (not shown) of the flexible sheath 308. The various components of the delivery device 300 can be formed and configured in the same manner as described above for delivery devices 100 and 200, and therefore, will not be described in detail below with reference to this embodiment.

The flexible shaft 302 includes a distal end portion 322 and a proximal end portion (not shown). The proximal end portion can optionally be coupled to a handle as described above. In this embodiment, the flexible shaft 302 defines a lumen 338 (see FIG. 9) that can receive a guidewire 339 therethrough. Thus, the delivery device 300 can be used with or without a guide catheter as described above. The retention element 306 is coupled to the distal end portion 322 of the flexible shaft 302 and is disposed at the distal end of the flexible shaft 302. The pushing element 304 is also coupled to the distal end portion 322 of the flexible shaft 302 and is disposed proximally from the retention element 306.

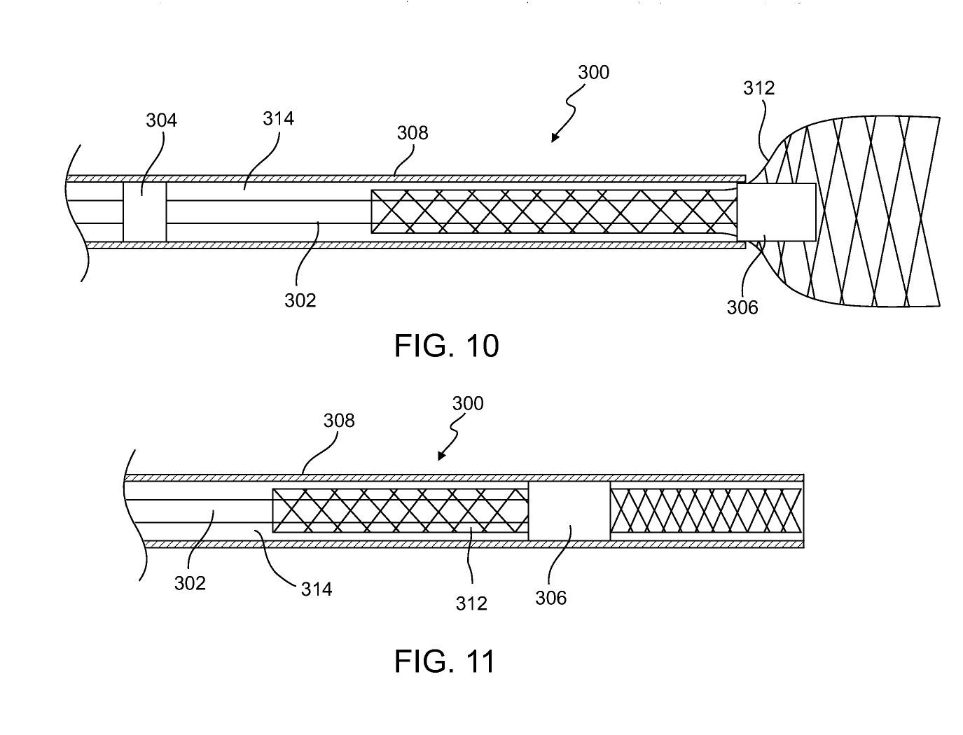

The delivery device 300 can be used to deliver and deploy a medical device 312 (see FIGS. 10-11) as described above for previous embodiments. The medical device 312 can be for example, a self-expanding intraluminal device. As described above, the medical device 312 can be placed within the lumen 314 of the flexible sheath 308 and positioned between the retention element 306 and the pushing element 304 as shown in FIG. 10.

In this example embodiment, the guidewire 339 can be inserted into a blood vessel and positioned at a desired treatment site. The lumen 338 of the flexible shaft 302 can be placed over the guidewire 339 and moved distally along the guidewire 339 until a distal end of the delivery device 300 is positioned at the desired location within the blood vessel. The guidewire 339 can then be removed from the patient's body.

To deploy the medical device 312 at the treatment site, the flexible shaft 302 can be moved or urged distally relative to the flexible sheath 308 such that the retention element 306 is moved distally outside of the distal end of the lumen 314 of the flexible sheath 308 and within the blood vessel, and the pushing element 304 contacts and moves the medical device 312 distally as described above. The flexible shaft 302 can be moved distally until the entire medical device 312 is fully deployed at the treatment site and the delivery device 300 can be removed in the same manner as described above with reference to FIG. 5. Alternatively, before the medical device 312 is fully deployed, the medical practitioner can recapture the medical device 312 and move the distal end of the delivery device 300 to a different location within the blood vessel, or remove the delivery device 300 and medical device 312 completely from the blood vessel.

In this embodiment, to recapture the medical device 312, the medical practitioner can move the flexible shaft 302 proximally relative to the flexible sheath 308 such that the retention element 306 is pulled or moved proximally through an interior of the deployed or expanded portion of the medical device 312 to a retention position, as shown in FIG. 10. In the retention position, at least a portion of the retention element 306 engages and forms a friction fit with an interior of the medical device 312. In this embodiment, the retention element 306 is sized such that as the flexible shaft 302 continues to be moved proximally, the retention element 306 moves proximally into the lumen 314 of the flexible sheath 308 and also pulls the medical device 312 within the lumen 314, as shown in FIG. 11. Thus, the outer perimeter or diameter of the retention element 306 is sufficiently large to engage a portion of an interior of the medical device 312 and sufficiently small such that the retention element 306 and medical device 312 can slidably move through the lumen 314 of the flexible sheath 308.

With the medical device 312 withdrawn within the flexible sheath 308, the delivery device 300 can be repositioned at a different location within the blood vessel to redeploy the medical device 312, or can alternatively be removed from the blood vessel. As described above for previous embodiments, the relative movement of the various components can alternatively be reversed. For example, the flexible sheath 308 can be moved relative to the flexible shaft 302.

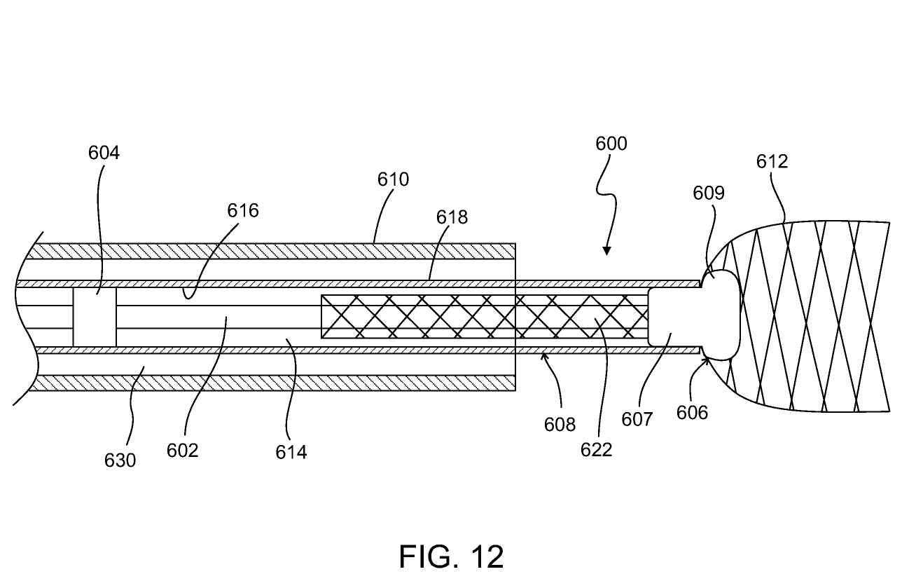

FIG. 12 illustrates another embodiment of a delivery system. A delivery system 600 includes a flexible sheath 608, a flexible shaft 602, a retention element 606 and a pushing element 604. The various components of the delivery device 600 can be formed and configured in the same manner as described above for previous embodiments. The flexible sheath 608 has an interior wall 616 defining a lumen 614 that extends between a distal end portion 618 and a proximal end portion (not shown) of the flexible sheath 608. The flexible shaft 602 includes a distal end portion 622 and a proximal end portion (not shown). The proximal end portion can optionally be coupled to a handle (not shown).

The retention element 606 is coupled to the distal end portion 622 of the flexible shaft 602 and is disposed at the distal end of the shaft 602. The pushing element 604 is also coupled to the distal end portion 622 of the flexible shaft 602 and is disposed proximally from the retention element 606.

In this embodiment, the delivery device 600 is shown coupled to, and used in conjunction with, a guide catheter 610. In FIG. 12, a distal portion of the delivery device 600 is shown partially disposed within a lumen 630 of the guide catheter 610 and partially extended distally outside the lumen 630. A self-expanding braided medical device 612 is shown disposed partially within the lumen 614 of the flexible sheath 608 at the distal end portion 618 of the flexible sheath 608, and partially extended distally outside the lumen 614.

In this embodiment, the retention element 606 has an hour glass or cork shape such that a first portion 607 of the retention element 606 has a diameter (or outer perimeter) sized to be disposed within the lumen 614 of the flexible sheath 608 and a second portion 609 that has a diameter (or outer perimeter) that is larger than a diameter of the lumen 614 such that the second portion 609 can not be moved inside the lumen 614. The process of deploying and recapturing the medical device 612 using the delivery system 660 can be performed in a similar manner as described above, for example, for delivery system 200 and, therefore, is not described in detail with reference to this embodiment. FIG. 12 illustrates the retention element 606 in a retention position, with the first portion 607 of the retention element 606 disposed partially within the lumen 614 of the flexible sheath 608. In alternative embodiments, a retention element can have an outer perimeter or outer diameter sized such that no part of the retention element fits within the lumen of the flexible sheath. Thus, in such an embodiment, the retention element can be pulled or positioned within a medical device (e.g., medical device 612) at a location proximate the distal end of the flexible sheath and will reside entirely outside the flexible sheath.

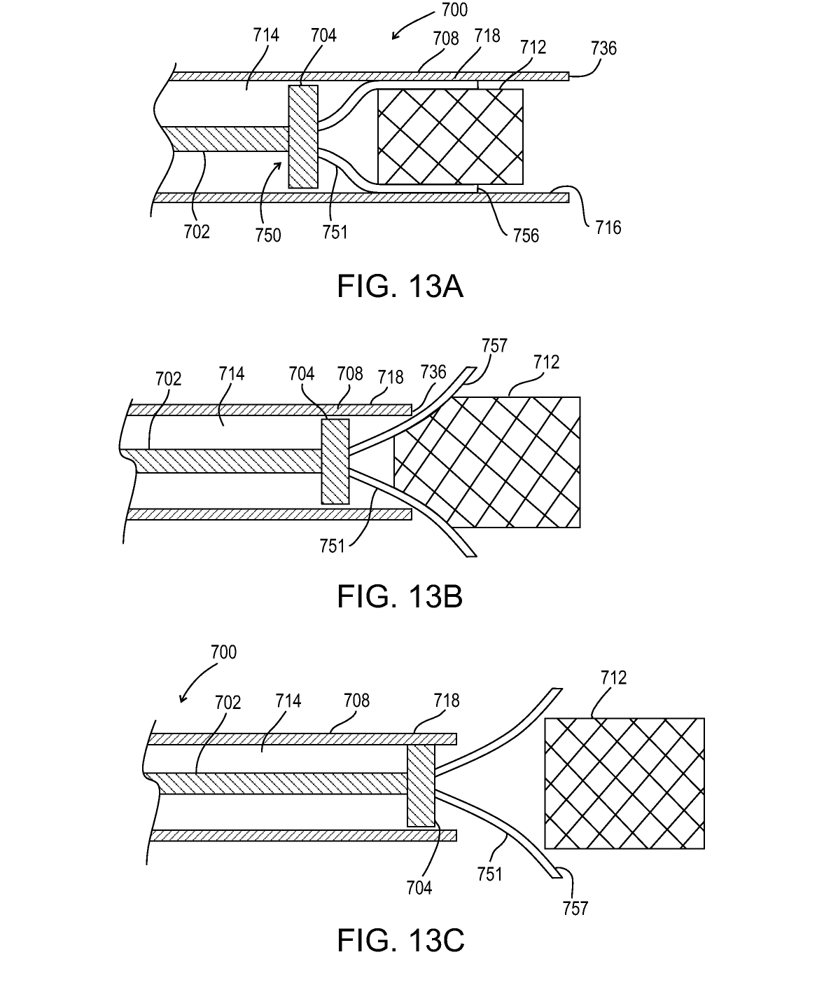

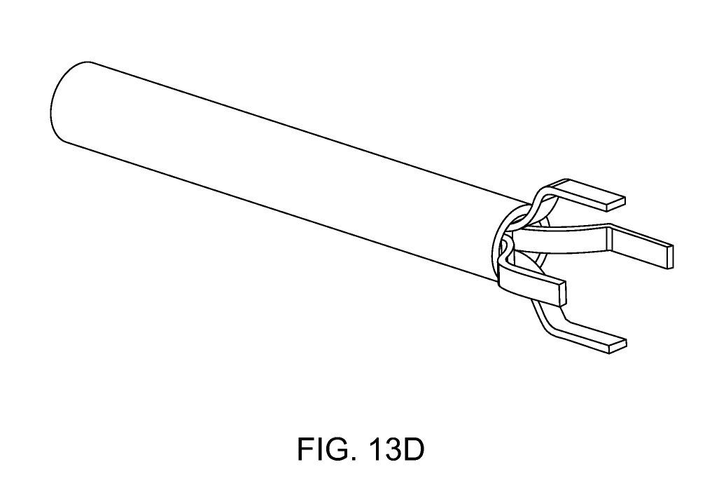

FIGS. 13A-13D illustrate another embodiment of a medical device delivery system. The delivery system 700 includes a flexible sheath 708 and a grip mechanism 750 to maneuver a medical device 712. The flexible sheath 708 has an interior wall 716 defining a lumen 714 that extends between a distal end portion 718 and a proximal end portion (not shown) of the flexible sheath 708. The various components of the delivery device 700 can be formed and configured in the same manner as described above for delivery devices 100, 200 and 300, and therefore, will not be described in detail below with reference to this embodiment.

The grip mechanism 750 includes a flexible shaft 702, a plurality of legs 751 and a pushing element 704. The grip mechanism 750 can optionally be coupled to a handle located at a proximal end of the sheath 708 via the flexible shaft 702. The handle can include one or more actuating mechanisms, such as, for example, a slide, a knob and/or a dial, configured to operate the grip mechanism 750 from a collapse configuration within the lumen 714 of the flexible sheath 708 (as shown in FIG. 13A) to a extended configuration outside the flexible sheath 708 (as shown in FIG. 13C).

The grip mechanism 750 includes at least two legs 751. Each of the legs 751 is attached to the pushing element 704 at their proximal ends and move with the pushing element 704. Said another way, the legs 751 are moveable with respect to the sheath 708 as the pushing element 704 is advanced or retracted in the lumen 714. The legs can be formed with any material such that they are sufficiently flexible to traverse tortuous blood vessels, to collapse within the lumen 714 and to expand outside side of the lumen 714, while having sufficient stiffness to be maneuverable within the blood vessel of a patient. For example, the legs can be made of any of a variety of materials, such as stainless steel, Nitinol, plastics, polymers and/or a combination thereof.

The inner surface 757 of the legs 751 is designed to frictionally engage the medical device 712 as the radial compression from the legs 751 increases and release the medical device 712 as the radial compression from the legs 751 decreases. The inner surface of the legs 751 can optionally be roughened by serrations, grinding or other surface treatment techniques. The cross-sectional shape of the legs 712 can be a rectangular, circular, D-shaped, or any other shape. It is to be understood that the legs can alternatively have other configurations, such as, for example, as shown in FIG. 13D where the delivery system has four legs coupled to the pushing element.

The delivery device 700 can be used to deliver and deploy a medical device 712 as described above for previous embodiments. The medical device 712 can be, for example, a self-expanding intraluminal device. As described above, the medical device 712 can be placed within the lumen 714 of the flexible sheath 708 and position between the legs 751 of the grip mechanism 750 as shown in FIG. 13A.

To deploy the medical device 712 at the treatment site, the distal end 736 of the sheath 708 is placed at the treatment site. The pushing element 704 can be moved or urged distally with the flexible shaft 702 relative to the flexible sheath 708. As the flexible shaft 702 and pushing element 704 are moved distally, the legs 751 with the medical device 712 are also moved distally until at least a portion of the medical device 712 is extended or deployed outside of the lumen 714 of the flexible sheath 708 and within the blood vessel, as shown in FIG. 13B. As the distal portion of the medical device 712 is deployed within the blood vessel, the medical device 712 can expand as it is removed from the radial compression of the legs 751. The medical practitioner can continue to move the flexible shaft 702 distally relative to the flexible sheath 708 until the entire medical device 712 is fully deployed or implanted at the treatment site, as shown in FIG. 13C.

Alternatively, before the medical device 712 is fully deployed as described above, the medical practitioner can recapture the medical device 712 and move the distal end 736 of the sheath 708 to a different location within the blood vessel, or remove them completely from the blood vessel. In this embodiment, to recapture the partially expanded medical device 712, the medical practitioner can move the sheath 708 distally relative to the shaft 702 such that it gradually compresses the legs 751 and the medical device 712 is retracted into the lumen 714 of the flexible sheath 708. Alternatively, the medical practitioner can move the shaft 702 proximally relative to the flexible sheath 708 such that the legs 751 with the medical device 712 form an interference fit with the flexible sheath 708 such that the flexible sheath 708 causes a radial compression on the legs 751 and the medical device 712. As the shaft 702 continues to move proximally relative to the flexible sheath 708, the legs 751 and the medical device 712 are radially compressed further within the lumen 714 of the flexible sheath 708. The proximal movement of the shaft 702 relative to the flexible sheath 708 will continue until the legs 751 and the medical device 712 are fully retracted within the lumen 714 of the flexible sheath 708, as shown in FIG. 13A. Once the medical device 712 is withdrawn within the flexible sheath 708, the delivery device 700 can be repositioned at a different location within the blood vessel to redeploy the medical device 712, or can alternatively be removed form the treatment site.

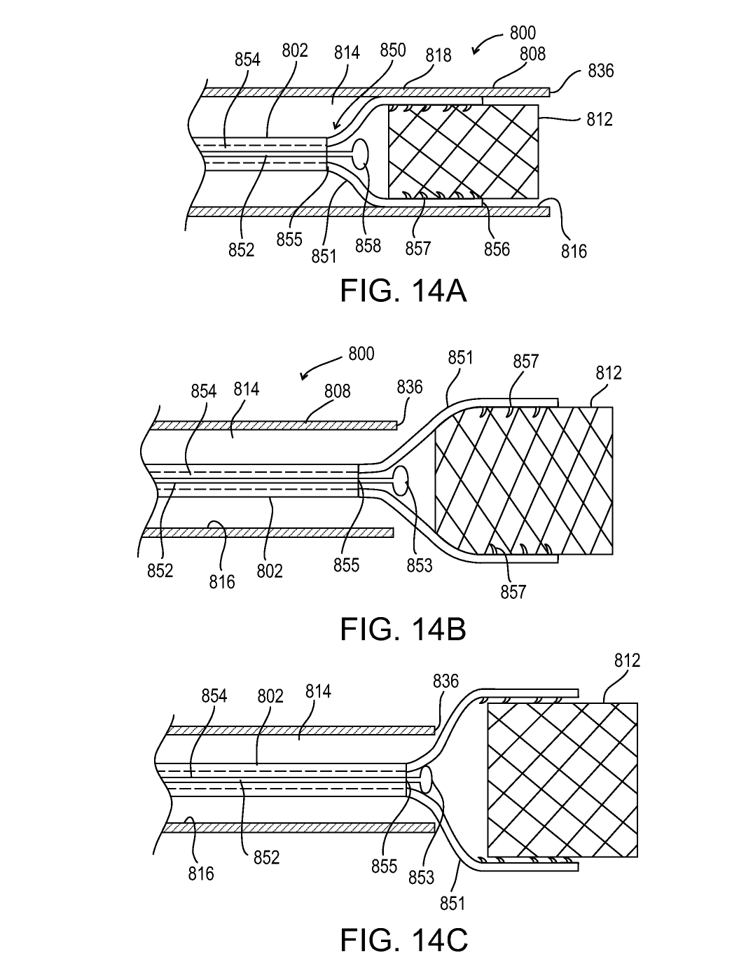

FIGS. 14A-14C illustrate another embodiment of a medical device delivery system. The delivery system 800 includes a flexible sheath 808 and a grip mechanism 850 to maneuver a medical device 812. The flexible sheath 808 has an interior wall 816 defining a lumen 814 that extends between a distal end portion 818 and a proximal end portion (not shown) of the flexible sheath 808. The various components except the delivery system 800, including the grip mechanism 850, can be formed and configured in the same manner as described above for delivery device 700, and therefore, will not be described in detail below with reference to this embodiment.

The grip mechanism 850 includes a flexible shaft 802, a plurality of legs 851, a cable 852 and a plunger 853. The grip mechanism 850 can optionally be coupled to a handle located at a proximal end of the sheath 808 via the flexible shaft 802 and the cable 852. The handle itself can include one or more actuating mechanisms, such as, for example, a slide, a knob and/or a dial to operate the grip mechanism 850 such as move the legs 851 from a collapse configuration within the lumen 814 of the sheath 808 (as shown in FIG. 14A) to a extended configuration outside the sheath 808 (as shown in FIG. 14C).

The grip mechanism 850 includes at least two legs 851. Each of the legs 851 is attached at their proximal end to the flexible shaft 802 and are movable with respect to the sheath 808 as the flexible shaft 802 is moved with respect to the sheath 808. The flexible shaft 802 and the cable 852 can be operated independently or together, for example, by actuating mechanisms on the handle. The plunger 853 is coupled to, and moves with, the cable 852 which is disposed in a lumen 854 of the flexible shaft 802. In this embodiment, the inner surface 856 of the legs 851 have barb-like inwardly protruded portions 857 to engage the medical device 812 during slidably movement of the legs 851 within and/or outside of the lumen 814 of the sheath 800.

The delivery device 800 can be used to deliver and deploy a medical device 812 as described above for previous embodiments. The medical device 812 can be, for example, a self-expanding intraluminal device. As described above, the medical device 812 can be placed within the lumen 814 of the flexible sheath 808 and positioned between the legs 851 of the grip mechanism 850 as shown in FIG. 14A.

To deploy the medical device 812 at the treatment site, the distal end 836 of the sheath 808 is placed at the treatment site. The flexible shaft 802 and the cable 852 can be moved or urged distally relative to the flexible sheath 808 such that the plunger 853 remains in its extended position relative to the flexible shaft 802 (as shown in FIG. 14A). As the flexible shaft 802 is moved distally, the legs 851 with the medical device 812 are also moved distally until at least a distal portion of the medical device 812 is extended or deployed outside of the lumen 814 of the flexible sheath 808 and within the blood vessel, as shown in FIG. 14B. As the legs 851 move outside of the flexible sheath 808, they can expand as they are removed from the radial compression of the sheath 808. Simultaneously, the medical device 812 can also expand as there is a reduction in the radial compression from the legs 851, although the legs 851 remain at least partially engaged with the medical device 812. The medical practitioner can continue to move the flexible shaft 802 distally relative to the flexible sheath 808 until the entire medical device 812 is fully outside of the flexible sheath 808. Once the medical device 812 is disposed at a desired treatment site such as blood vessel, the medical device 812 can be fully deployed by retracting the plunger 853 from the extended position to the withdrawn position, thereby exerting enough radial tension on the legs to open them further to remove the radial compression of the legs 851 on the medical device 812. Alternatively, before the medical device 812 is fully deployed, the medical practitioner can recapture the medical device 812 and move the distal end 836 of the sheath 808 to a different location within the blood vessel, or remove them completely from the blood vessel as described above.

FIG. 15 illustrates a method of using a delivery device as described herein to deliver and deploy a medical device according to an embodiment. For example, the method can be used to dispose in a blood vessel adjacent an aneurysm a self-expanding medical device. At 450, a distal end of the delivery device is positioned near the treatment site. For example, the delivery device can include a sheath having a distal end and an interior wall defining an internal lumen and a flexible shaft having a proximal portion and a distal portion. A distal end of the sheath can be positioned near the treatment site. At 452, the sheath is moved proximally relative to the shaft, or the shaft is moved distally relative to the sheath, to a partially deployed position in which a retention element disposed on a distal end portion of the shaft is a sufficient distance distally from the distal end of the sheath to allow a portion of the medical device (mounted on the flexible shaft and constrained within the sheath in a radially compressed configuration) to self-expand beyond the distal end of the sheath and to engage an inner wall of the blood vessel near the treatment site.

At 454, the flexible shaft is partially retracted such that the medical device is frictionally engaged between the retention element and the sheath. At 456, the sheath, flexible element, and medical device can be moved proximally within the blood vessel.

FIG. 16 illustrates another method of using a delivery device as described herein to deliver and deploy a medical device according to an embodiment. At 550, positioning a distal end of a catheter at a desired treatment site. A 552, moving a delivery device through a lumen of the catheter until a distal end of the delivery device is disposed near the treatment site. For example, the delivery device can include a sheath having a distal end and an interior wall defining an internal lumen and a flexible shaft having a proximal portion and a distal portion. A distal end of the sheath can be positioned near the treatment site. At 554, the sheath of the delivery device is moved proximally relative to the shaft, or the shaft is moved distally relative to the sheath. to a partially deployed position in which a retention element disposed on a distal end portion of the shaft is a sufficient distance distally from the distal end of the sheath to allow a portion of the medical device (mounted on the flexible shaft and constrained within the sheath in a radially compressed configuration) to self-expand beyond the distal end of the sheath and to engage an inner wall of the blood vessel near the treatment site.

At 556, the catheter can be moved distally relative to the sheath and flexible shaft, or the flexible shaft and sheath can be moved proximally relative to the catheter, so as to partially constrain at least a portion of the medical device within the catheter. At 558, the sheath, flexible element, and medical device can be moved proximally within the blood vessel.

The various components of a delivery device and guide catheter, as described herein can be formed with one or more of a variety of different biocompatible materials. For example, a guide catheter can be formed with, for example, a polycarbonate portion and include a nylon proximal portion reinforced with a stainless steel braid, and a Pebax distal portion reinforced with a stainless steel coil. A guide catheter can also include, for example, a PTFE (Teflon) inner liner, a platinum/iridium marker band in a distal tip portion, and/or a lubricious coating on a portion distal portion (e.g., 12-15 cm of the distal end of the catheter). In some embodiments, all or a portion of a delivery device can be formed with, for example, polycarbonate, stainless steel, nylon, polyethylene, PTFE lined polyimide, Nitinol, polyimide, PTFE (Teflon), and/or polyurethane. The delivery device can also include, for example, a platinum/iridium marker portion, cyanoacrylate glue, and/or UV cured adhesive, and/or other suitable adhesives used for medical devices.

While various embodiments of the invention have been described above, it should be understood that they have been presented by way of example only, and not limitation. Where methods and steps described above indicate certain events occurring in certain order, those of ordinary skill in the art having the benefit of this disclosure would recognize that the ordering of certain steps may be modified and that such modifications are in accordance with the variations of the invention. Additionally, certain of the steps may be performed concurrently in a parallel process when possible, as well as performed sequentially as described above. The embodiments have been particularly shown and described, but it will be understood that various changes in form and details may be made.

For example, although various embodiments have been described as having particular features and/or combinations of components, other embodiments are possible having any combination or sub-combination of any features and/or components from any of the embodiments described herein. The various combinations of components can be included in a kit, as described herein. The specific configurations of the various components can also be varied. For example, the size and specific shape of the various components can be different than the embodiments shown, while still providing the functions as described herein.

In addition, although the delivery system (100, 200, 300) and methods were shown and described in conjunction with the deployment of a self-expanding braided medical device (112, 212, 312), the delivery systems (100, 200, 300) and methods described herein can be used to deliver and deploy other types of medical devices. In addition, although the delivery system (100, 200, 300) and methods were described for use in treating intracranial pathologies, such as an aneurism, the delivery system and methods can be used in other areas of the body and other blood vessels.

What is claimed is:

1. A delivery system for a medical device, comprising:
a flexible sheath having an interior wall bounding a lumen, said lumen having a distal end; and
a flexible shaft having a proximal portion and a distal portion a retention element and a pushing element are each disposed on the distal portion of the flexible shaft, said pushing element being spaced proximally from said retention element,
said flexible shaft, retention element, and pushing element being disposable within said lumen of said flexible sheath to collectively define with said interior wall a region sized to contain the medical device,
said flexible shaft being movable distally through said lumen between a stowed position in which said retention element is proximal to said distal end of said lumen and a deployed position in which said pushing element at least partially extends distally from said distal end of said lumen,
said flexible shaft being movable proximally through said lumen between said deployed position and a partially deployed position in which said retention element is proximate to said distal end of said lumen,
said retention element being configured to form with the medical device an interference fit with said flexible sheath when said flexible shaft is urged proximally from said partially deployed position to a retention position to engage the medical device with said flexible sheath.

2. The delivery system of claim 1, wherein the medical device is expandable from a stored configuration in which the medical device has a stored outer perimeter smaller than a perimeter of said lumen of said flexible sheath to a deployed configuration having a deployed outer perimeter larger than the stored outer perimeter.

3. The delivery system of claim 1, wherein said retention element is resilient.

4. The delivery system of claim 1, wherein an outer perimeter of said retention element is sufficiently large to engage a portion of the medical device and sufficiently small such that said retention element and the medical device can slidably move through said lumen of said flexible sheath.

5. The delivery system of claim 3, wherein the resilient material of said retention element enables said retention element to be maneuvered within a blood vessel and form said interference fit with said flexible sheath.

6. The delivery system of claim 1, wherein said flexible shaft defines a guidewire lumen configured to receive a guidewire.

7. The delivery system of claim 1, wherein said retention element has a hardness rating in a range of about 30 to 90 on the Shore D scale.

8. The delivery system of claim 1, wherein said retention element has a hardness rating in a range of about 30 to 90 on the Shore A scale.

9. The delivery system of claim 1, wherein said flexible sheath has a hardness rating in a range of about 30 to 90 on the Shore D scale.

10. The delivery system of claim 1, wherein said flexible sheath has a hardness rating in a range of about 30-90 on the Shore A scale.

11. The delivery system of claim 1, wherein the flexible sheath further includes a stiffening member.

12. The delivery system of claim 11, wherein said stiffening member is a helical coil.

13. The delivery system of claim 1, wherein said flexible sheath has an outer diameter in a range of about 0.5 mm to about 3.0 mm.

14. A kit, comprising:
a delivery device including a sheath having a distal end and an interior wall bounding a lumen and a flexible shaft having a proximal portion and a distal portion, said flexible shaft including a retention element and a pushing element disposed on said distal portion and being disposable within said lumen, and
a medical device mountable on said flexible shaft and configured to be constrained within said sheath in a radially compressed configuration,
said flexible shaft being movable distally through said lumen between a stowed position in which said retention element is proximal to said distal end of said lumen and a deployed position in which said medical device at least partially extends distally from said distal end of said lumen,
said flexible shaft being movable proximally through said lumen between said deployed position and a partially deployed position in which said retention element is proximate to said distal end of said lumen,
said retention element being configured to form with said medical device an interference fit with said sheath when said flexible shaft is urged proximally from said partially deployed position to a retention position to engage said medical device with said sheath.

15. The kit of claim 14, wherein said medical device is a mesh tubular body.

16. The kit of claim 15, wherein said mesh tubular body is formed by braiding a plurality of filaments.

17. The kit of claim 14, wherein said flexible shaft, retention element, and pushing element collectively define with said interior wall a region sized to contain said medical device.

18. The kit of claim 14, wherein said lumen of said sheath has a diameter sufficiently sized to movably receive therein said medical device in at least one of a relaxed configuration or a partially relaxed configuration in which said medical device has an outer perimeter less than an outer perimeter of said medical device when said medical device is deployed outside of said lumen.

19. A method for disposing in a blood vessel at a treatment site adjacent an aneurysm a self-expanding medical device using a sheath having a distal end and an interior wall defining an internal lumen and a flexible shaft having a proximal portion and a distal portion, the flexible shaft including a retention element disposed at the distal portion and being movable through the lumen, the medical device being mounted on the flexible shaft and constrained within the sheath in a radially compressed configuration, the method comprising:
positioning the distal end of the sheath near the treatment site;
moving the sheath proximally relative to the flexible shaft to a partially deployed position in which the retention element is sufficient distance distal from the distal end of the sheath to allow a portion of the medical device to self-expand beyond the distal end of the sheath to engage an inner wall of the blood vessel near the treatment site; and
partially retracting the flexible shaft so as to frictionally engage the medical device between the retention element and the sheath.

20. The method of claim 19, further comprising:
moving the sheath, flexible element, and medical device proximally within the blood vessel.

21. The method of claim 19, further comprising:
introducing the sheath into a catheter, the sheath being movable through the catheter; and
positioning a distal end of the catheter near the treatment site.

22. The method of claim 21, further comprising:
moving the catheter distally relative to the sheath and flexible shaft so as to partially constrain at least a portion of the medical device within the catheter.

23. The method of claim 22, further comprising:
moving the sheath, retention element, and medical device within the blood vessel.

24. The method of claim 21, further comprising:
moving the flexible shaft and sheath proximally relative to the catheter so as to partially constrain at least a portion of the medical device within the catheter.

25. The method of claim 24, further comprising:
moving the sheath, flexible element, and medical device within the blood vessel.

26. The method of claim 19, further comprising:
prior to positioning the distal end of the sheath, placing the medical device within the internal lumen of the sheath at a spaced distance from a distal end of the sheath.
